(19) United States
(12) Patent Application Publication
(10) Pub. No.: US 2019/0388664 A1
(43) Pub. Date: Dec. 26, 2019

(54) Control Method Of Local Release For Target Compounds By Using Patterning Hydrogel To Nanoporous Membrane

(71) Applicant: KNU-Industry Cooperation Foundation, Gangwon-Do (KR)

(72) Inventor: Kwang Ho LEE, Seoul (KR)

(21) Appl. No.: 16/453,529

(22) Filed: Jun. 26, 2019

(30) Foreign Application Priority Data
Jun. 26, 2018 (KR) 10-2018-0073184

(51) Int. Cl. A61M 31/00; C08J 3/24; C08J 3/075

(52) U.S. Cl. CPC A61M 31/002; C08J 3/24; B82Y 5/00; A61M 2207/00; C08J 3/075

(57) ABSTRACT

Provided is a method of controlling local release of target compounds by patterning a hydrogel carrying a bone morphogenetic protein as the target compounds on a nanoporous membrane. The nanoporous membrane is capable of controlling local release of the bone morphogenetic protein as a carrier of the bone morphogenetic protein while performing a basic function of the membrane of preventing infiltration of connective tissue, and thus, there is an advantage in that the nanoporous membrane can facilitate generation of controlled bone in a local region.

### CROSS-REFERENCE TO RELATED APPLICATION

This application claims benefit of and priority to Korean Patent Application No. 10-2018-0073184, filed on 26 Jun. 2018. The entire disclosure of the applications identified in this paragraph are incorporated herein by reference.

### FIELD

The present invention relates to a method of controlling local release of target compounds by patterning a hydrogel on a nanoporous membrane.

### BACKGROUND

In the field of dentistry and orthopedics in the related art, for formation and reconstruction of a bone tissue which has been damaged, bone morphogenetic proteins have been used. These materials can only be used for regeneration and treatment of the damaged tissue if the materials are released locally in a human body. However, in the current techniques, there have been used methods of synthesizing carriers or nanoparticles in a drug in a polymeric-based fibrous membrane and attaching the carriers or nanoparticles or immobilizing attaching the carriers or nanoparticles by injection to a desired tissue region. However, because the growth of bone may be induced in an undesired tissue region and delivery of the drug is not quantitative, the technique has difficulties in correct treatment for bone formation.

The following Table 1 lists the types of bone morphogenetic proteins used for the regeneration of damaged bone.

TABLE 1

| Signaling molecule | Phenotype |
|---|---|
| BMP1 | Die after birth, failure of ventral body wall closure |
| BMP2 | Embryonically lethal, defects in amnion/chorion and cardiac |
| | development; limb: spontaneous fractures and impaired fracture |
| | repair8; chondrocyte: severe chondrodysplasia; cardiac progenitor: |
| | abnormal heart valve development38; myocardium: defects in |
| | myocardial patterning |
| BMP3 | Increased bone density |
| BMP4 | Embryonically lethal, lack of mesoderm formation, no PGCs, no lens |
| | induction; heterozygotes: various organ abnormalities; hypomorph: |
| | AVCD, HSC microenvironment defect; limb bud mesoderm: |
| | defective digit patterning; adipocyte: enlarged adipocytes and |
| | impaired insulin sensitivity; other targeted: loss-of-trachea |
| | phenotype, abnormal branchial arch arteries and outflow tract |
| | septation, defects in mandibular development, defects in vestibular |
| | apparatus |
| BMP5 | Short ear phenotype; smaller and weaker bones |
| BMP6 | Delay in sternum ossification; smaller long bones; decreased fertility |
| BMP7 | Die after birth, defects in kidney and eye development; defects in |
| | skeletal patterning; impaired corticogenesis; decreased brown fat, |
| | diminished Langerhans cell number; inducible deletion: precocious |
| | differentiation of kidney progenitor cells; limb: no effect; podocyte: |
| | defective kidney development |
| BMP8 | Germ cell degeneration; defective PGC formation; germ cell |
| | deficiency and infertility |
| BMP9/GDF2 | Abnormal lymphatic development |
| BMP10 | Reduced cardiomyocyte proliferation |
| BMP11/GDF11 | Die after birth, defects in A-P patterning; smaller pancreas; reduced |
| | β-cell numbers; kidney agenesis; slower spinal cord neuron |
| | differentiation; increased olfactory neurogenesis; retinal |
| | abnormalities |
| BMP12/GDF7 | Increased endochondral bone growth; smaller bone cross-sectional |
| | parameters; no effect on tail tendon phenotype; subtle effects on |
| | Achilles tendon; defective dorsal interneuron formation; sterile with |
| | seminal vesicle defects |
| BMP13/GDF6 | Bone fusions in wrists and ankles; accelerated coronal suture |
| | fusion; eye and neural defects; Klippel-Feil syndrome; males: lower |
| | tail tendon collagen |
| BMP14/GDF5 | Brachypodism; malformations in bones of limb, sternum, and digits; |
| | delayed fracture healing; impaired joint formation and osteoarthritis; |
| | weaker Achilles tendon; increased scarring after myocardial |
| | infarction; altered skin properties |
| BMP15 | Males: normal and fertile; females: subfertile with decreased |
| | fertilization and ovulation rates |

In addition, in order to regenerate the damaged bone, it is necessary to use a membrane to prevent the formation of scar tissue by blocking a connective tissue from infiltrating into the damaged region for a certain period of time. However, in the techniques, only the blocking of infiltration of the connective tissue into an existing membrane is is performed (in FIG. 1, a schematic diagram of a restoration process and a membrane for regenerating a bone is illustrated).

Therefore, in the present invention, the delivery of the bone morphogenetic protein and the function of the membrane are simultaneously performed.

### SUMMARY

### Technical Problem

According to many clinical researches, it is known that a bone morphogenetic protein is directly helpful for regeneration of damaged bone tissue and formation of bone. Many studies have been actively made to develop a delivery system for the bone morphogenetic protein.

Meanwhile, in the related art, a method of delivering the bone morphogenetic protein have performed by using a form of a hydrogel, a microsphere, a nanoparticle, a fiber, and the like configured with a material such as a metal, a ceramic, a polymer, and a composite. In addition, these materials are intended to be dissolved in a desired site. However, there is a problem called an ectopic growth, and there is a limit in the quantitative, localized delivery of the bone morphogenetic protein.

### Solution to Problem

In order to solve the above-described problems, the present invention is to provide a method of controlling local release of target compounds by patterning a hydrogel on a nanoporous membrane.

Preferably, the present invention provides a method of controlling local release by patterning a hydrogel on a nanoporous membrane, wherein the target compounds contain a bone morphogenetic protein or a drug.

In addition, preferably, the present invention provides a method of controlling local release by patterning a hydrogel on a nanoporous membrane, wherein the nanoporous membrane is any one of a biodegradable nanoporous membrane and a non-biodegradable nanoporous membrane.

In addition, more preferably, the present invention provides a method of controlling local release by patterning a hydrogel on a nanoporous membrane, wherein the nanoporous membrane is manufactured by an electrospinning process.

In addition, more preferably, the present invention provides a method of controlling local release by patterning a hydrogel on a nanoporous membrane, wherein the hydrogel contains at least one of gelatin methacryloyl (gel-MA), hyaluronic acid, and Na-alginate.

In addition, more preferably, the present invention provides a method of controlling local release by patterning a hydrogel on a nanoporous membrane, including steps of: (S1) preparing a micromold having a plurality of concave grooves; (S2) pouring a hydrogel solution into the micromold; (S3) filling the plurality of concave grooves on the micromold with the hydrogel solution; (S4) covering a semi-permeable nanoporous membrane on the micromold filled with the hydrogel solution; (S5) cross-linking the hydrogel to the micromold covered with the nanoporous membrane; (S6) detaching the micromold from the semi-permeable nanoporous membrane; and (S7) forming a hydrogel micropattern on the semi-permeable nanoporous membrane.

More preferably, the present invention provides a method of controlling local release by patterning a hydrogel on a nanoporous membrane, wherein the cross-linking in the step (S5) is performed by any one of a photo cross-linking method using light or an ion cross-linking method using ion exchange.

In addition, more preferably, the present invention provides a method of controlling local release by patterning a hydrogel on a nanoporous membrane, wherein the micromold of step (S1) is made of any one of polydimethylsiloxane (PDMS), Teflon, and polymethylmethacrylate (PMMA).

On the other hand, the present invention also provides a nanoporous membrane manufactured by a method including steps: (S1) preparing a micromold having a plurality of concave grooves; (S2) pouring a hydrogel solution into the micromold; (S3) filling the plurality of concave grooves on the micromold with the hydrogel solution; (S4) covering a semi-permeable nanoporous membrane on the micromold filled with the hydrogel solution; (S5) cross-linking the hydrogel to the micromold covered with the nanoporous membrane; (S6) detaching the micromold from the semi-permeable nanoporous membrane; and (S7) forming a hydrogel micropattern on the semi-permeable nanoporous membrane.

### Advantageous Effects of Invention

According to the present invention, it is possible to control local release of a bone morphogenetic protein by using a nanoporous membrane which can serve as a carrier of the bone morphogenetic protein while performing a basic function of the membrane. The bone morphogenetic protein is essentially used in orthopedic and dental fields, but the delivery method is not quantitative and causes a lot of side effects. However, in the present invention, a delivery method and process capable of performing local release quantitatively can be applied in the clinical field. In addition, it is also expected that the present invention can be used for a case where quantitative release of a drug as well as a bone morphogenetic protein inside and outside a human body is required.

### BRIEF DESCRIPTION OF DRAWINGS

FIGS. 1A, 1B, 1C and 1D are diagrams illustrating a commonly used restoration process and a nanoporous membrane for regeneration of bone.

FIG. 2 illustrates a carrier of a bone morphogenetic protein in hydrogel and a patterning process on a nanoporous membrane.

FIG. 3 illustrates a carrier of a bone morphogenetic protein in hydrogel and a pattern on a nanoporous membrane according to the present invention.

FIGS. 4A and 4B illustrates a photograph (a) of an intaglio PDMS mold replicated with PDMS and an intaglio PDMS mold covered with a nanoporous membrane and a photograph (b) of a cross section of the intaglio PDMS mold.

FIGS. 5A, 5B and 5C illustrates photographs of a hydrogel (a) patterned on a nanoporous membrane and a three-dimensional patterned hydrogel (b) imaged by a confocal laser microscope.

FIGS. 6A, 6B, 6C, 6D, 6E, 6F, 6G, 6H and 6I illustrate charts obtained by tracking intensities of fluorescence attenuated by release of a fluorescent material in hydrogel by using a fluorescent material in order to verify release control performance of a bone morphogenetic protein according to the present invention.

FIG. 7 illustrates release control values verified by using a bone morphogenetic protein and a hydrogel concentration.

FIG. 8 illustrates observation of skeleton change of MG63 cells by immobilization of a bone morphogenetic protein and local release.

FIG. 9 illustrates observation of calcification of MG63 cells by immobilization of a bone morphogenetic protein and local release.

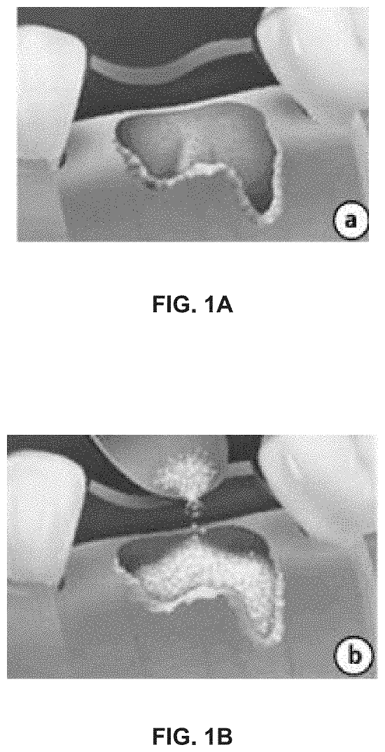

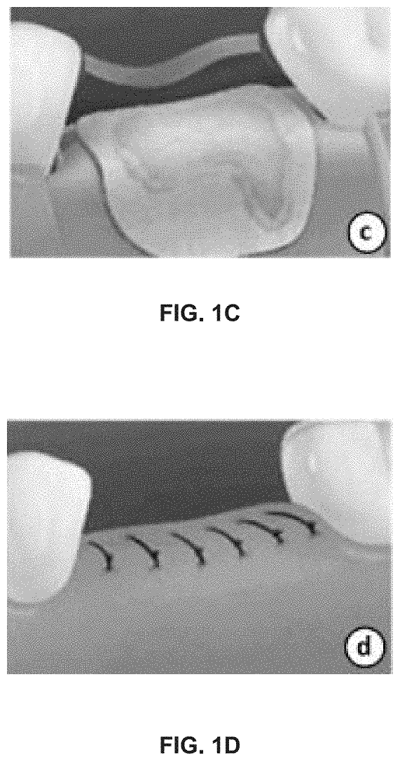

### DETAILED DESCRIPTION

Hereinafter, a preferred embodiment of the present invention will be described in detail with a manufacturing process.

It should be noted that the specific numerical values given as examples are only for explaining the technical idea of the present invention in more detail, and that the technical idea of the present invention is not limited thereto and that various modifications are possible.

In addition, in the specification of the present invention, the same components are denoted by the same reference numerals, and those components which are well known in the technical field and can be easily created by the ordinary skilled in the art will be omitted in detailed description.

The present invention provides a method of controlling local release of target compounds by patterning a hydrogel on a nanoporous membrane.

Preferably, the present invention provides a method of controlling local release by patterning a hydrogel on a nanoporous membrane, wherein the target compounds contain a bone morphogenetic protein or a drug.

In addition, preferably, the present invention provides a method of controlling local release by patterning a hydrogel on a nanoporous membrane, wherein the nanoporous membrane is any one of a biodegradable nanoporous membrane and a non-biodegradable nanoporous membrane.

In addition, more preferably, the present invention provides a method of controlling local release by patterning a hydrogel on a nanoporous membrane, wherein the nanoporous membrane is manufactured by an electrospinning process.

In addition, more preferably, the present invention provides a method of controlling local release by patterning a hydrogel on a nanoporous membrane, wherein the hydrogel contains at least one of gelatin methacryloyl (gel-MA), hyaluronic acid, and Na-alginate.

In addition, more preferably, the present invention provides a method of controlling local release by patterning a hydrogel on a nanoporous membrane, including steps of: (S1) preparing a micromold having a plurality of concave grooves; (S2) pouring a hydrogel solution into the micromold; (S3) filling the plurality of concave grooves on the micromold with the hydrogel solution; (S4) covering a semi-permeable nanoporous membrane on the micromold filled with the hydrogel solution; (S5) cross-linking the hydrogel to the micromold covered with the nanoporous membrane; (S6) detaching the micromold from the semi-permeable nanoporous membrane; and (S7) forming a hydrogel micropattern on the semi-permeable nanoporous membrane.

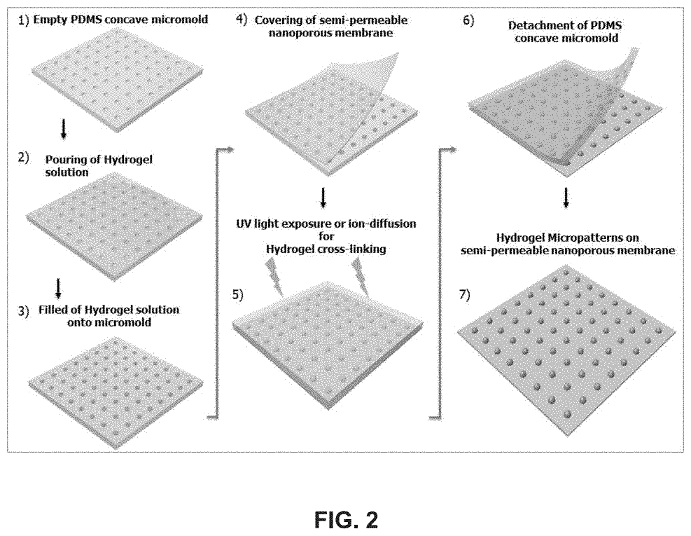

The carrier of the bone morphogenetic protein in hydrogel and the patterning process on the nanoporous membrane are illustrated in FIG. 2.

More preferably, the present invention provides a method of controlling local release by patterning a hydrogel on a nanoporous membrane, wherein the cross-linking in the step (S5) is performed by any one of a photo cross-linking method using light or an ion cross-linking method using ion exchange.

In addition, more preferably, the present invention provides a method of controlling local release by patterning a hydrogel on a nanoporous membrane, wherein the micromold of step (S1) is made of any one of polydimethylsiloxane (PDMS), Teflon, and polymethylmethacrylate (PMMA).

On the other hand, the present invention also provides a nanoporous membrane manufactured by a method including steps: (S1) preparing a micromold having a plurality of concave grooves; (S2) pouring a hydrogel solution into the micromold; (S3) filling the plurality of concave grooves on the micromold with the hydrogel solution; (S4) covering a semi-permeable nanoporous membrane on the micromold filled with the hydrogel solution; (S5) cross-linking the hydrogel to the micromold covered with the nanoporous membrane; (S6) detaching the micromold from the semi-permeable nanoporous membrane; and (S7) forming a hydrogel micropattern on the semi-permeable nanoporous membrane.

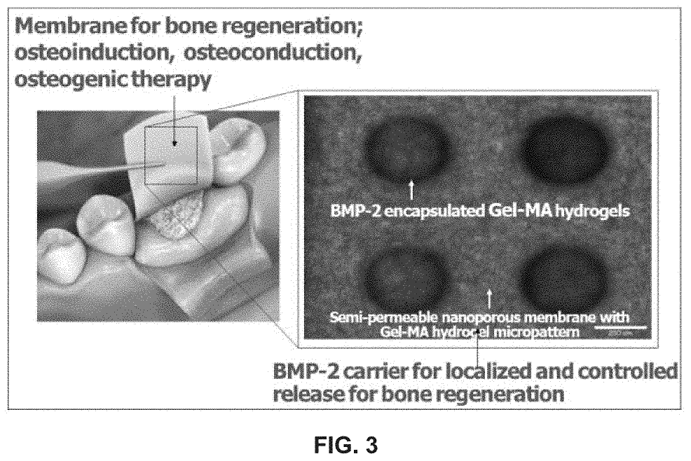

FIG. 3 illustrates the carrier of the bone morphogenetic protein in hydrogel and the pattern on the nanoporous membrane according to the present invention.

The present invention provides a method of manufacturing a semi-permeable nanoporous membrane, including: (S1) preparing a micromold having a plurality of concave grooves; (S2) pouring a hydrogel solution into the micromold; (S3) filling the plurality of concave grooves on the micromold with the hydrogel solution; (S4) covering the semi-permeable nanoporous membrane on the micromold filled with the hydrogel solution; (S5) cross-linking the hydrogel to the micromold covered with the nanoporous membrane through light irradiation or ion diffusion; (S6) detaching the micromold from the semi-permeable nanoporous membrane; and (S7) forming a hydrogel micropattern on the semi-permeable nanoporous membrane.

The micromold may be made of polydimethylsiloxane (PDMS), Teflon, or polymethylmethacrylate (PMMA).

Particularly, in the present invention, nanoporous membranes are manufactured according to biodegradable and non-biodegradable methods by using biopolymers of polyurethane and polylactide-co-glycolide (PLGA), which have been approved by the US Food and Drug Administration, for in vivo transplantation. The nanoporous membrane is manufactured by an electrospinning process.

In addition, for local release of the bone morphogenetic protein, patterning by using cross-linking of hydrogel with excellent biocompatibility is used. In the present invention, a the cross-linking method, there are used a photo cross-linking method using light and an ion cross-linking method using ion exchange. The material used was hydrogel patterned with gelatin methacryloyl (gel-MA), hyaluronic acid, and Na-alginate. The hydrogel patterning is performed by using the bone morphogenetic protein contained in the hydrogel in accordance with each condition.

In addition, for the patterning, a master mold for supporting hydrogel is required. On the other hand, various master molds are manufactured through a soft-lithography process and a 3D printing process. From the manufactured master mold (intaglio), a replica mold (embossing) is manufactured by using a photomicrograph (PDMS) with excellent biocompatibility and excellent optical transparency. The above-mentioned hydrogel is inserted into the replica mold formed as an embossing mold. A nanoporous membrane manufactured by electrospinning is covered with the mold. The hydrogel is formed by light transmission and ion exchange, and thus, various patterns containing the bone morphogenetic protein is manufactured.

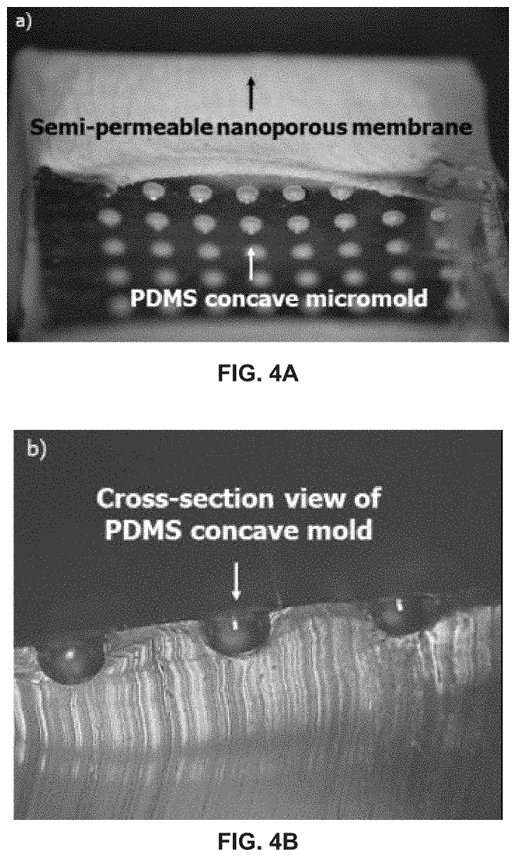

This can be confirmed in FIG. 4 illustrating a photograph (a) of an intaglio PDMS mold replicated with PDMS and an intaglio PDMS mold covered with a nanoporous membrane and a photograph (b) of a cross section of the intaglio PDMS mold.

The concentration of the bone morphogenetic protein used can be selected widely depending on the shape, size, and type of the pattern.

In addition, with respect to the membrane, the release rate of the carried drug can be also controlled by using a biodegradable membrane and a non-biodegradable membrane.

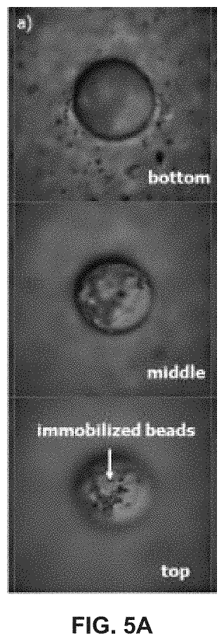

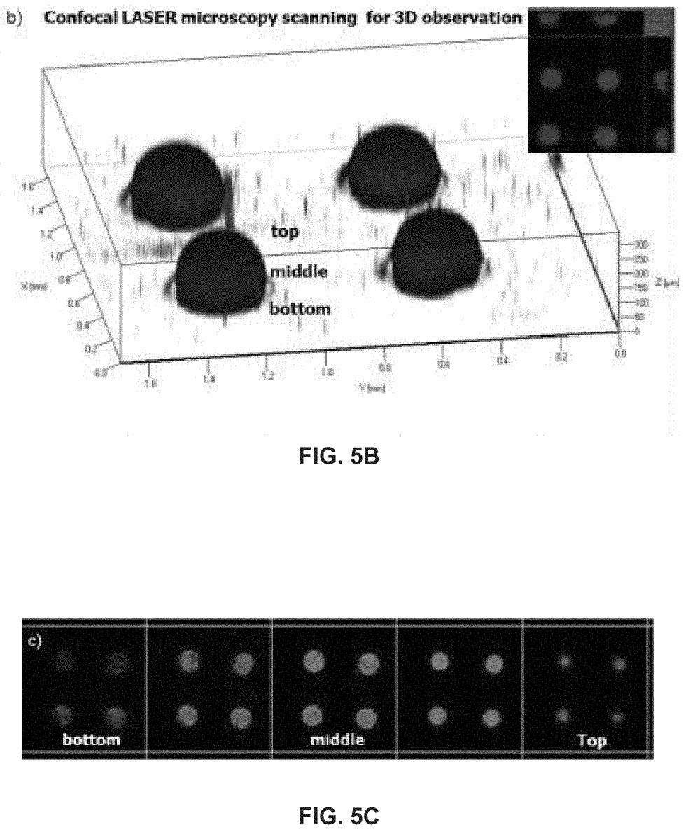

FIG. 5 illustrates photographs of a hydrogel (a) patterned on a nanoporous membrane and a three-dimensional patterned hydrogel (b) imaged by a confocal laser microscope.

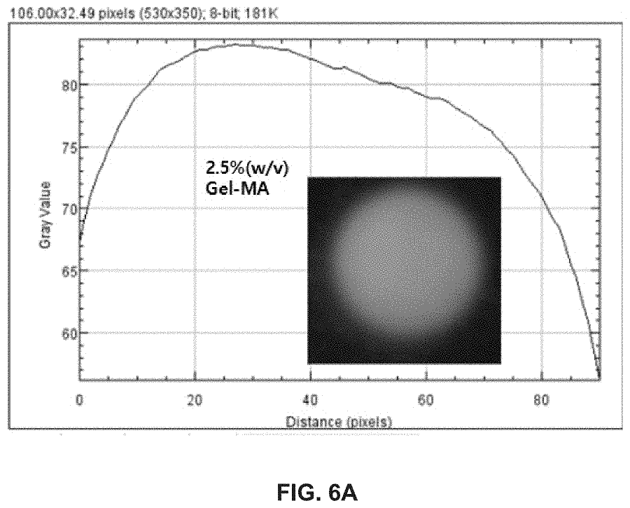

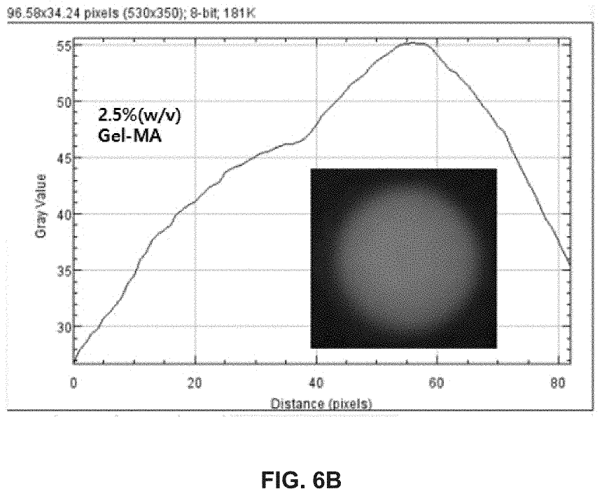

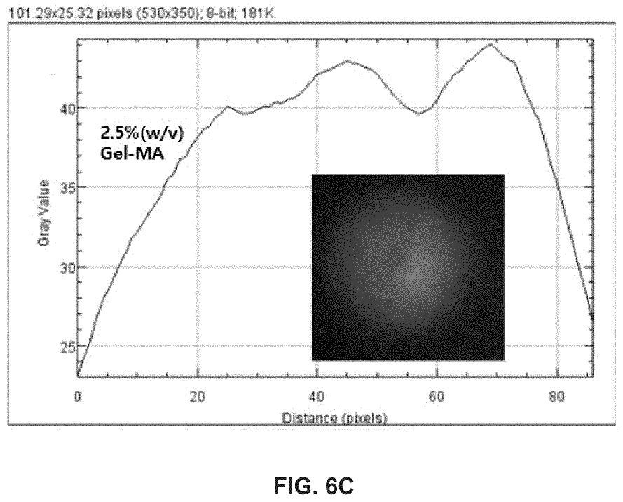

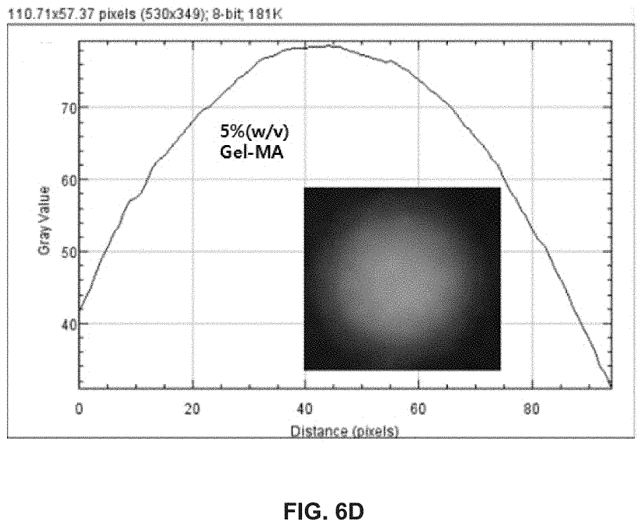

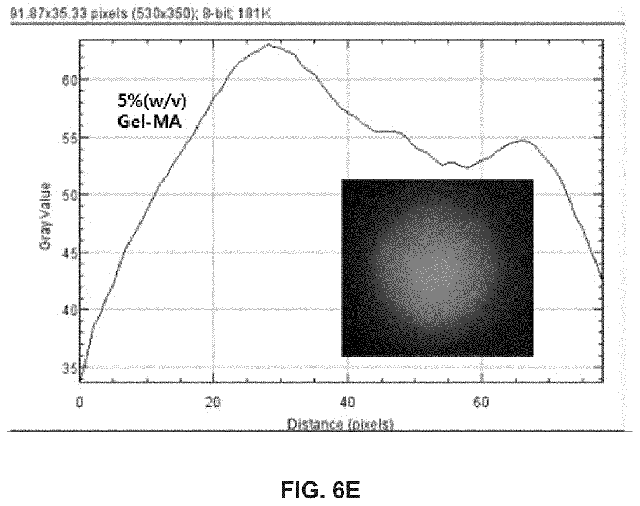

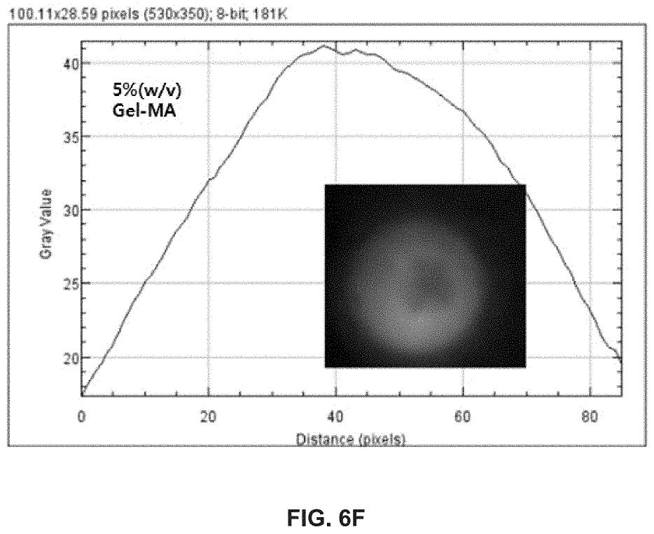

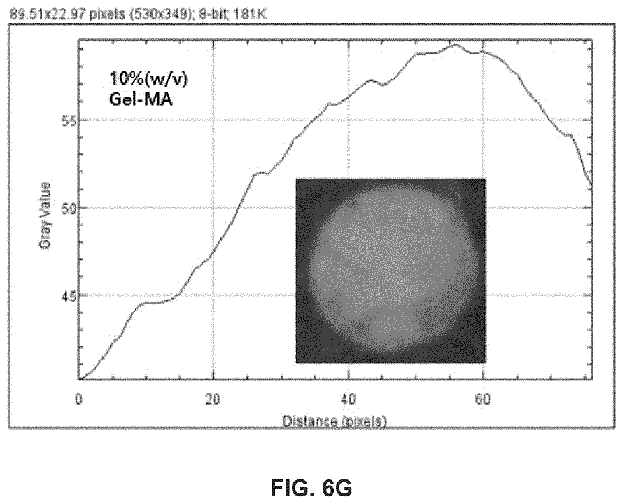

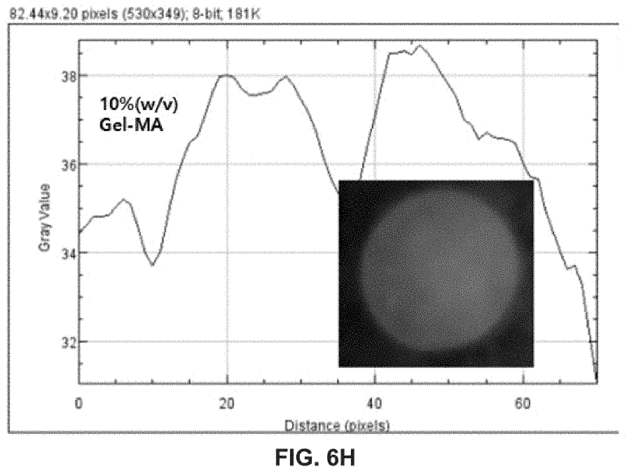

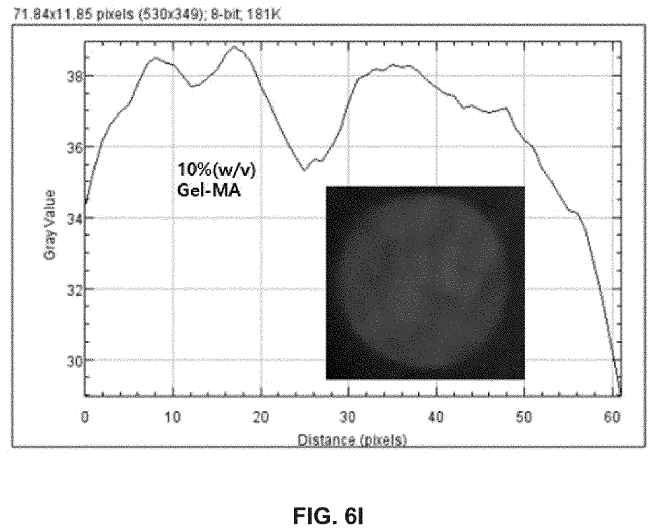

FIG. 6 illustrates charts obtained by tracking intensities of fluorescence attenuated by release of a fluorescent material in hydrogel by using a fluorescent material in order to verify release control performance of a bone morphogenetic protein according to the present invention.

In detail, patterning is performed by using a fluorescent material of fluorescence FITC-BSA (70 kDa) with the concentration of hydrogel being 2.5, 5, and 10% (w/v), and the intensity of the fluorescence attenuated along with the release of the fluorescent material in the hydrogel is tracked for six days.

It can be seen that, in a) to c) of FIG. 6, since the concentration of the hydrogel is low, the intensity of the fluorescence is relatively high, and the intensity is relatively rapidly weakened. In can be seen that, in g) and h) of FIG. 6, since the concentration of hydrogel is high, the intensity of the fluorescence is also weak at the beginning, and since the concentration of the released hydrogel is low, the intensity of the fluorescence is also decreased at a relatively low rate.

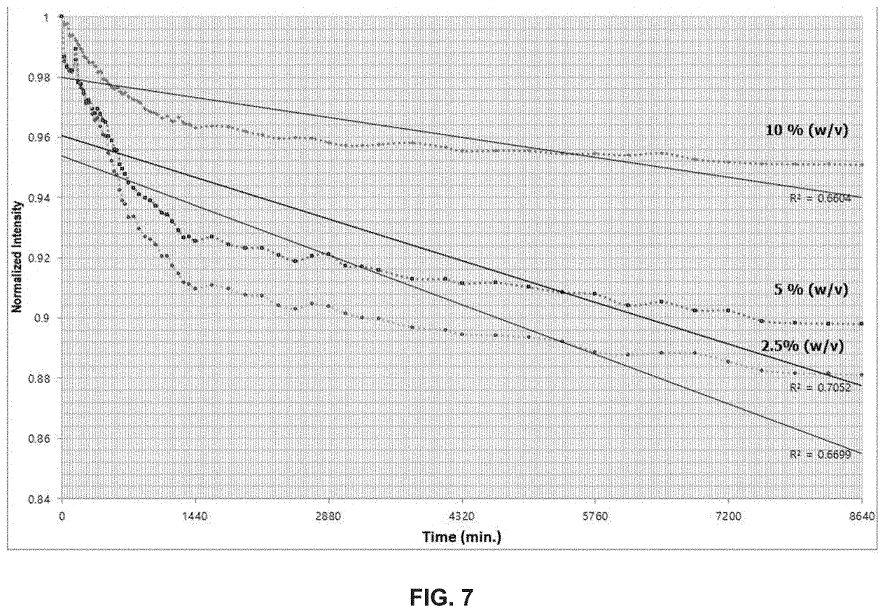

FIG. 7 illustrates release control values verified by using a bone morphogenetic protein and a hydrogel concentration.

In detail, it is expended that, the higher the concentration of the hydrogel, the lower the release rate of the drug, and the lower the concentration of the hydrogel, the higher the release rate of the drug. In addition, it is expected that the release rate depends on the concentration of the bone morphogenetic protein.

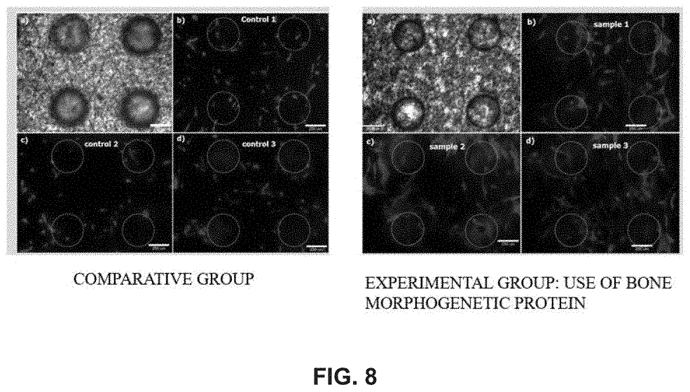

FIG. 8 illustrates observation of skeleton change of MG63 cells by immobilization of the bone morphogenetic protein and the local release.

In the comparative group, since there is no release of the bone morphogenetic protein in comparison with the experimental group, it is observed that the skeleton of the cells is not developed relatively. In the experiment group, it is observed that, a lot of cells proliferate around the pattern where the bone morphogenetic protein is immobilized around the pattern, and the skeleton is greatly developed.

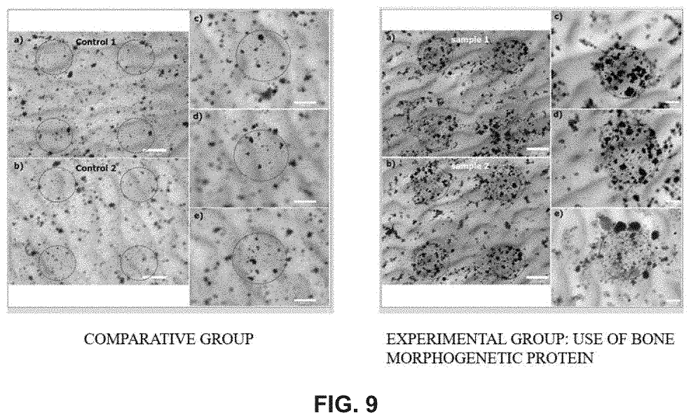

FIG. 9 illustrates observation of calcification of MG63 cells by immobilization of the bone morphogenetic protein and the local release.

In contrast, in the comparative group, since there is no release of the bone morphogenetic protein in comparison with the experimental group, it is observed that the calcification of the cells is not progressed relatively. In the experiment group, it is observed that the calcification of the cells is greatly progressed around the pattern where the bone morphogenetic protein is immobilized around the pattern.

Due to the development of the delivery method capable of controlling local release of a bone morphogenetic protein through a hydrogel on a semi-permeable nanoporous membrane used in the present invention and the manufacturing method thereof, it is possible to simultaneously realize localized and quantitative release of the bone morphogenetic protein for bone regeneration and the effect of membrane to prevent the infiltration of connective tissue used in existing clinic fields such as orthopedics and dentistry, it is expected that new applications to the existing clinic and a rapid entry into the market can be achieved. Fundamental technologies and products having both local delivery and release functions of membranes, such as bone morphogenetic proteins and drugs, have not yet been disclosed in the world. Therefore, it is essential to secure the fundamental technologies.

In addition, the fundamental technology disclosed in the present invention has not yet been reported in academic or industrial fields. Above all, the local delivery of the bone morphogenetic protein and the drug and the effect of the membrane that can prevent the infiltration of connective tissue can be achieved simultaneously. Therefore, in the fields such as orthopedics, dentistry, and dermatology, the possibility of transferring technology to medical companies and pharmaceutical companies is very high.

What is claimed is:

1. A method of controlling local release of target compounds by patterning a hydrogel on a nanoporous membrane.

2. The method according to claim 1, wherein the target compounds contain a bone morphogenetic protein or a drug.

3. The method according to claim 1, wherein the nanoporous membrane is any one of a biodegradable nanoporous membrane and a non-biodegradable nanoporous membrane.

4. The method according to claim 1, wherein the nanoporous membrane is manufactured by an electrospinning process.

5. The method according to claim 1, wherein the hydrogel is contains at least one of gelatin methacryloyl (gel-MA), hyaluronic acid, and Na-alginate.

6. The method according to claim 1, wherein the patterning of the hydrogel on the nanoporous membrane includes:
(S1) preparing a micromold with a plurality of concave grooves;
(S2) pouring a hydrogel solution into the micromold;
(S3) filling the plurality of concave grooves on the micromold with the hydrogel solution;
(S4) covering a semi-permeable nanoporous membrane on the micromold filled with the hydrogel solution,
(S5) cross-linking the hydrogel to the micromold covered with the nanoporous membrane;
(S6) detaching the micromold from the semi-permeable nanoporous membrane; and
(S7) forming a hydrogel micropattern on the semi-permeable nanoporous membrane.

7. The method according to claim 6, wherein the cross-link of step (S5) is performed by a photo cross-linking method using light or an ion cross-linking method using ion exchange.

8. The method according to claim 6, wherein the micromold of the step (S1) is made of any one of polydimethylsiloxane (PDMS), Teflon, and polymethylmethacrylate (PMMA).

9. A nanoporous membrane manufactured by a method including steps of:
(S1) preparing a micromold with a plurality of concave grooves;
(S2) pouring a hydrogel solution into the micromold;
(S3) filling the plurality of concave grooves on the micromold with the hydrogel solution;
(S4) covering a semi-permeable nanoporous membrane on the micromold filled with the hydrogel solution,
(S5) cross-linking the hydrogel to the micromold covered with the nanoporous membrane;
(S6) detaching the micromold from the semi-permeable nanoporous membrane; and
(S7) forming a hydrogel micropattern on the semi-permeable nanoporous membrane.
